(19) United States
(12) Patent Application Publication
(10) Pub. No.: US 2008/0026481 A1
(43) Pub. Date: Jan. 31, 2008

(54) Molecular based two-marker assays that predict outcome of adenocarcinoma patients

(76) Inventor: Michael Mitas, Monks Corner, SC (US)

Correspondence Address: NEEDLE & ROSENBERG, P.C., SUITE 1000, 999 PEACHTREE STREET, ATLANTA, GA, 30309-3915, US

(21) Appl. No.: 11/820,092

(22) Filed: Jun. 18, 2007

Related U.S. Application Data
(60) Provisional application No. 60/814,365, filed on Jun. 16, 2006.

(51) Int. Cl. G01N 33/00

(52) U.S. Cl. 436/86

(57) ABSTRACT

Provided is a method of identifying a subject at risk of recurrence of adenocarcinoma, comprising detecting markers associated with recurrence. Provided is a method of identifying a subject at risk for recurrence of adenocarcinoma, comprising determining the ratio of EpCAM2 to CK19 in primary adenocarcinoma tissue from the subject, a high ratio of EpCAM2 to CK19 M indicating a subject at risk for recurrence. Also provided is method of identifying a subject at risk for recurrence of adenocarcinoma, comprising determining the ratio of CK19 and P-cadherin in primary adenocarcinoma tissue from the subject, a low ratio of CK19 to P-cadherin indicating a subject at risk for recurrence. A method of identifying a subject at risk for recurrence of adenocarcinoma is provided, comprising determining the ratio of Map7 to EpCAM2 in primary adenocarcinoma tissue from the subject, a low ratio of Map7 to EpCAM2 indicating a subject at risk for recurrence. Provided is a method of identifying a subject at risk for recurrence of adenocarcinoma, comprising determining the ratio of P-cadherin to E-cadherin in primary adenocarcinoma tissue from the subject, a high ratio of P-cadherin to E-cadherin indicating a subject at risk for recurrence.

### CROSS REFERENCE TO RELATED APPLICATIONS

This application claims the benefit of priority of U.S. Provisional Application No. 60/814,365, filed Jun. 16, 2006, which application is incorporated herein by this reference in its entirety.

### BACKGROUND

A significant number of patients recur after resection of stage I and II adenocarcinomas. For example, despite surgical resection, patients with pathologic stage I non-small cell lung cancer (NSCLC) will have an approximately 30-40 percent incidence of recurrence and those with stage II a 45% to 60% recurrence rate.¹ The development of metastatic disease is the most common cause of death among NSCLC patients and results from dissemination of malignant cells. At present the standard of care is to administer postoperative adjuvant chemotherapy to those patients with stage II NSCLC.^{2,3} Although there was initial enthusiasm for administering adjuvant therapy to resected stage IB patients, recent data do not support this practice.³⁻⁵ However, subsets of stage I patients could potentially benefit from further treatment to prevent recurrence; likewise a method to predict which stage II patients could avoid the unnecessary toxicity of chemotherapy would be helpful.

It is now recognized that the ability of cells to gain metastatic potential is an intrinsic property of the primary tumor.^{6,7} The ability to predict clinical outcome based on analysis of primary tumors would allow cancer patients to be treated more effectively. Tools used to predict recurrence have included immunohistochemical analysis,¹² cDNA microarray profiling,¹³⁻¹⁷ real-time reverse transcription polymerase chain reaction (RT-PCR),^{6,18-20} and most recently, proteomics.²¹ However, the problem with many of these expression studies is that they require measurements of large sets of predictive genes using a platform (cDNA microarray analysis) that is not well suited to clinical application. Thus, what is needed is a method of predicting clinical outcome of resected early stage adenocarcinoma by measuring the expression of a few critically important genes.

### SUMMARY OF THE INVENTION

Provided is a method of identifying a subject at risk of recurrence of adenocarcinoma, comprising detecting markers associated with recurrence.

### BRIEF DESCRIPTION OF THE FIGURES

FIG. 1 is a bar graph showing the relationship of P-cadherin to E-cadherin in primary versus metastatic tissue.

FIG. 2 is a correlation map of cancer-associated genes. Correlation map of the genes was constructed as described in the text. Genes are positioned in a hypothetical cell to reflect intracellular, membrane-bound, or extracellular localization. The thickness of a solid line connecting a given gene pair is ˜proportional to the R² value of gene expression, which ranges from 0.91 (p=1.9×10⁻²⁴) for the Spint1/SNC19 pair, to 0.55 (p=7.2×10⁻⁶) for the TFF1/S100P pair.

FIG. 3 is a Kaplan Meier survival analysis. Data generated from single marker (FIG. 3A) and CK19/EpCAM2 (FIG. 3B) analyses.

### DETAILED DESCRIPTION OF THE INVENTION

Provided is a method of predicting clinical outcome in patients with adenocarcinoma, comprising measuring the amount of EpCAM2 and the amount of cytokeratin 19 in primary tumor tissue from the patient, a high ratio of EpCAM2 to CK19 indicating shorter term survival of the patient. In this method, a high ratio of EpCAM2 to CK19 is >128 or within a range of >8 to >2048.

Provided is a method of identifying a subject at risk for recurrence of adenocarcinoma, comprising determining the ratio of EpCAM2 to CK19 in primary adenocarcinoma tissue from the subject, a high ratio of EpCAM2 to CK19 M indicating a subject at risk for recurrence. In this method, a high ratio of EpCAM2 to CK19 is >128 or within a range of >8.0 to >128 or within a range of >8 to >2048.

Also provided is a method of identifying the presence of metastatic adenocarcinoma tissue in a subject, comprising measuring EpCAM2 and CK19 in primary adenocarcinoma tissue from the subject, a high ratio of EpCAM2 to CK19 indicating the presence of metastatic adenocarcinoma tumor tissue in the subject. In this method, a high ratio of EpCAM2 to CK19 is >128 or within a range of >8.0 to >128 or within a range of >8 to >2048.

Provided is a method of predicting clinical outcome in patients with adenocarcinoma, comprising measuring the amount of cytokeratin 19 and the amount of P-cadherin in primary tumor tissue from the patient, an amount of cytokeratin 19 that is higher than the amount of P-cadherin indicating longer term survival of the patient than an amount of cytokeratin 19 that is lower than the amount of P-cadherin. In this method, a low ratio of CK19 to P-cadherin is >0.5 or in a range of >8 to >0.03.

Also provided is a method of predicting clinical outcome in patients with adenocarcinoma, comprising measuring the amount of cytokeratin 19 and the amount of P-cadherin in primary tumor tissue from the patient, the greater the difference in amounts of cytokeratin 19 and P-cadherin indicating a shorter survival term than amounts of cytokeratin 19 and P-cadherin that are similar. In this method, a low ratio of CK19 to P-cadherin is >0.5 or in a range of >8 to >0.03.

Also provided is method of identifying a subject at risk for recurrence of adenocarcinoma, comprising determining the ratio of CK19 and P-cadherin in primary adenocarcinoma tissue from the subject, a low ratio of CK19 to P-cadherin indicating a subject at risk for recurrence. In this method, a low ratio of CK19 to P-cadherin is >0.5 or in a range of >8 to >0.03.

A method of identifying the presence of metastatic adenocarcinoma tissue in a subject is provided, comprising measuring CK19 and P-cadherin in primary adenocarcinoma tissue from the subject, a low ratio of CK19 to P-cadherin indicating the presence of metastatic adenocarcinoma tissue in the subject.

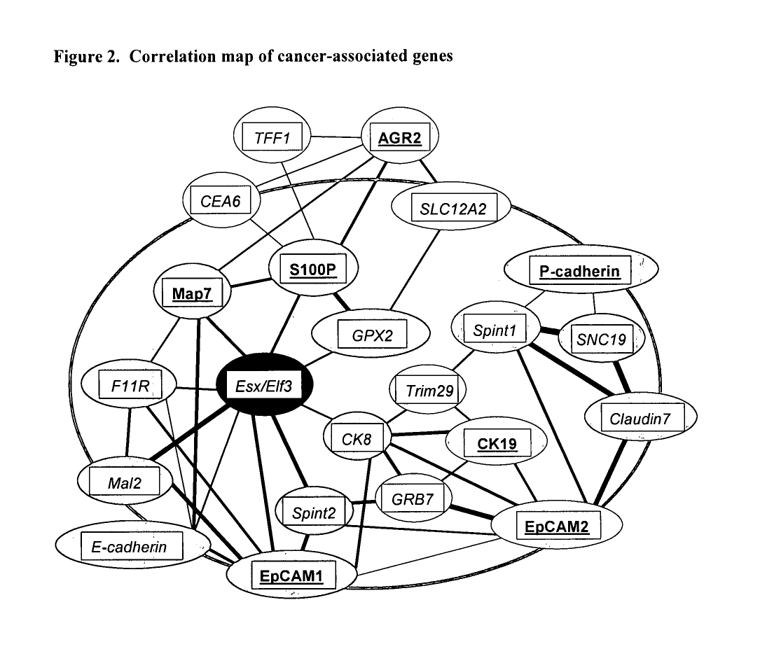

Examples of differences in amount of cytokeratin 19 and the amount of P-cadherin in primary tumor tissue that are indicative of likely metastasis and or shorter-term survival are shown in FIG. 2.

Provided is a method of identifying a subject at risk for recurrence of adenocarcinoma, comprising determining the ratio of Map7 to EpCAM2 in primary adenocarcinoma tissue from the subject, a low ratio of Map7 to EpCAM2 indicating a subject at risk for recurrence. In this method, the ratio with prognosis of high risk is 16:1 and the ration with prognosis of long term survival is 128:1.

A method of identifying the presence of metastatic adenocarcinoma tissue in a subject is provided, comprising measuring Map7 and EpCAM2 in primary adenocarcinoma tissue from the subject, a low ratio of Map7 to EpCAM2 indicating the presence of metastatic adenocarcinoma tissue in the subject.

Provided is a method of predicting clinical outcome in patients with adenocarcinoma, comprising measuring the amount of Map7 and the amount of EpCAM2 in primary tumor tissue from the patient, an amount of Map7 that is higher than the amount of Ep-CAM2 indicating longer term survival of the patient than an amount of Map7 that is lower than the amount of EpCAM2.

Provided is a method of identifying a subject at risk for recurrence of adenocarcinoma, comprising determining the ratio of P-cadherin to E-cadherin in primary adenocarcinoma tissue from the subject, a high ratio of P-cadherin to E-cadherin indicating a subject at risk for recurrence. Biological samples from a subject containing pancreatic, colon and esophageal cancer have elevated ratios of E-cadherin to P-cadherin gene expression. In this method a high ratio is from about 5:2 to about 5:3.

Also provided method of identifying primary adenocarcinoma tumor tissue, comprising measuring P-cadherin and E-cadherin in the tumor tissue, a low ratio of P-cadherin to E-cadherin indicating the presence of primary adenocarcinoma tumor tissue. In the disclosed method, a low ratio is from about 2:5 to about 3:5.

Further provided is a method of identifying the presence of metastatic adenocarcinoma tissue in a subject, comprising measuring P-cadherin and E-cadherin in primary adenocarcinoma tissue from a subject, a high ratio of P-cadherin to E-cadherin indicating the presence of metastatic adenocarcinoma tissue. In this method a high ratio is from about 5:2 to about 5:3.

In the methods of determining risk of recurrence, the risk of recurrence is based on the presence of the disclosed recurrence-associated marker ratios in the primary tissue. The presence of the recurrence-associated marker ratios in the primary tumor tissue indicates that metastasis from the primary tumor has already occurred at the time the primary tumor was collected or that the primary tumor is aggressive and recurrence at the primary site is likely to occur and to metastasize.

In the disclosed methods the adenocarcinoma can be non small cell lung cancer (NSCLC). In the disclosed methods the adenocarcinoma can be pancreatic cancer, colon cancer or esophageal cancer, or another adenocarcinoma listed below. In the disclosed methods, the primary tumor tissue is adenocarcinoma. For example, the adenocarcinoma can be lung tumor tissue. In the disclosed method of identifying metastatic tumor tissue, the primary tumor tissue can be adenocarcinoma. For example, the adenocarcinoma can be lung tumor tissue. In the disclosed method, the metastatic tissues can be from pancreatic, colon and esophageal cancer patients.

In the methods of predicting clinical outcome, the longer term survival is considered to be greater than 2 years. In the methods of predicting clinical outcome, non-recurrence during a period of greater than 4 years is an even stronger indicator of long term survival.

Disclosed are methods of identifying patients who are candidates for further anti-cancer therapy following surgery to remove primary tumors.

In one aspect, the method calls for measuring P-cadherin and E-cadherin in tumor tissue from the subject, a high ratio of P-cadherin to E-cadherin indicating the presence of metastatic tumor tissue, thus indicating the need for further anti-cancer therapy.

In one aspect, the method calls for measuring EpCAM2 and CK19 in tumor tissue from the subject, a high ratio of EpCAM2 to CK19 indicating the presence of metastatic adenocarcinoma tumor tissue in the subject, thus indicating the need for further anti-cancer therapy.

In one aspect, the method calls for measuring CK19 and P-cadherin in tumor tissue from the subject, a low ratio of CK19 to P-cadherin indicating the presence of metastatic adenocarcinoma tissue in the subject, thus indicating the need for further anti-cancer therapy.

In one aspect, the method calls for measuring Map7 and EpCAM2 in tumor tissue from the subject, a low ratio of Map7 to EpCAM2 indicating the presence of metastatic adenocarcinoma tissue in the subject, thus indicating the need for further anti-cancer therapy.

In the disclosed method of identifying patients in need of further anti-cancer therapy, the primary tumor tissue can be adenocarcinoma. For example, the adenocarcinoma can be lung tumor tissue. In the disclosed method, the metastatic tissues can be from pancreatic, colon and esophageal cancer patients.

The types of further treatment that can be administered are treatments for adenocarcinomas that are known in the art. These treatments can be selected for use with patients identified as needing further therapy. For example, the list below is contemplated as part of the present disclosure.

Antineoplastic compounds: Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Other anti-neoplastic compounds include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; atrsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance genie inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfmosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

Anti-cancer Supplementary Potentiating Agents include: Tricyclic anti-depressant drugs (e.g., imipramine, desipramine, amitryptyline, clomiprainine, trimipramine, doxepin, nortriptyline, protriptyline, amoxapine and maprotiline); non-tricyclic anti-depressant drugs (e.g., sertraline, trazodone and citalopram); Ca.sup.++ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL. The compounds of the invention also can be administered with cytokines such as granulocyte colony stimulating factor.

Disclosed is a method of treating adenocarcinoma in a subject, comprising inhibiting the expression of P-cadherin in the subject. The inhibition can be via administration of P-cadherin antibodies, small molecules targeted to P-cadherin or other pharmaceutical interventions such as those described herein.

Disclosed is a method of treating adenocarcinoma in a subject, comprising inhibiting the expression of EpCAM2 in the subject. The inhibition can be via administration of EpCAM2 antibodies, small molecules targeted to EpCAM2 or other pharmaceutical interventions such as those described herein.

By “treatment” is meant a method of reducing the effects of a disease or condition. Treatment can also refer to a method of reducing the disease or condition itself rather than just the symptoms. The treatment can be any reduction from native tumor levels and can be but is not limited to the complete ablation of the disease, condition, or the symptoms of the disease or condition. For example, a disclosed method for treating adenocarcinoma is considered to be a treatment if there is a 10% reduction in one or more indicators of the disease in a subject with the disease when compared to precancer levels in the same subject or control subjects. Thus, the reduction in P-cadherin level or tumor load can be a 10, 20, 30, 40, 50, 60, 70, 80, 90, 100%, or any amount of reduction in between as compared to native or control levels.

For each of the herein described markers or marker pairs, provided is an assay using single markers or a combination of markers that allows the determination of whether an individual has adenocarcinoma.

As used in the specification and the appended claims, the singular forms “a,” “an” and “the” include plural referents unless the context clearly dictates otherwise. Thus, for example, reference to “a nucleic acid” includes mixtures of two or more such nucleic acids, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that these data, represent endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

“Specific” nucleic acids are nucleic acids that are associated with a particular gene, protein-coding sequence or functional nucleic acid, such that the nucleic acid does not bind in a detectable manner with other nucleic acids.

A “subject” is an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and more preferably, a human.

“Non-primary or secondary tissue” is tissue in which the cancer did not first arise and to which the cancer metastasized, or spread, from the primary tissue.

As used herein, “overexpression” means expression greater than the expression detected in normal, non-cancerous tissue. For example, a nucleic acid that is overexpressed may be expressed about 1 standard deviation above normal, or about 2 standard deviations above normal, or about 3 standard deviations above the normal level of expression. Therefore, a nucleic acid that is expressed about 3 standard deviations above a normal, control level of expression (as determined in non-cancerous tissue) is a nucleic acid that is overexpressed.

Provided is a method for detecting micrometastases (occult metastases/metastases) of adenocarcinomas in a subject, comprising detecting in non-primary/secondary tissue of the subject overexpression of P-cadherin, the overexpression of P-cadherin in non-primary/secondary tissue being correlated with micrometastases (occult metastases/metastases) of adenocarcinoma in the subject.

Provided is a method for detecting micrometastases (occult metastases/metastases) of adenocarcinomas in a subject, comprising detecting in non-primary/secondary tissue of the subject overexpression of EpCAM2, the overexpression of EpCAM2 in non-primary/secondary tissue being correlated with micrometastases (occult metastases/metastases) of adenocarcinoma in the subject.

In the disclosed methods, adenocarcinoma means a carcinoma of glandular origin in any tissue having glandular tissue. To be classified as adenocarcinoma, the cells do not necessarily need to be part of a gland, as long as they have secretory properties. This form of carcinoma can occur in some higher mammals as well as man. The adenocarcinoma can be selected from the group consisting of breast; bladder, colon; esophageal; pancreatic; prostate, stomach; calcifying epithelial odontogenic tumor (CEOT); appendiceal; oral cavity; hepatocellular carcinoma.

In the disclosed methods, the non-primary/secondary tissue/metastatic tissue can be axillary lymph node, sentinel lymph node, mediastinal lymph node, other lymph nodes, bone marrow, or peripheral blood. Furthermore, because the present data show the correlation of the overexpression of certain markers in non-primary cancer tissue with metastasis of the cancer, the invention provides a method of detecting metastasis to other tissues. For example, bone marrow (e.g., aspirates), blood, bone and adipose tissue, among others, can be tested for the overexpression of the markers described herein, as well as for other markers that become associated with adenocarcinoma. Similarly, other nucleic acids that are now known to be associated with epithelial cell cancer, or are later found to be associated with epithelial cancer, can be used in the methods described herein. These tissues can be isolated using any available method including the methods disclosed herein.

Provided is a composition comprising a pair of primers specific for a nucleic acid encoding cytokeratin 19. Provided is a composition comprising a pair of primers specific for a nucleic acid encoding EpCAM2. Provided is a composition comprising a pair of primers specific for a nucleic acid encoding Map7. Also provided is a composition comprising a pair of primers specific for a nucleic acid encoding E-cadherin. Also provided is a composition comprising a pair of primers specific for a nucleic acid encoding P-cadherin. Also provided is a composition comprising a pair of primers specific for cytokeratin 19 and a pair of primers specific for P-cadherin. Also provided is a composition comprising a pair of primers specific for cytokeratin 19 and a pair of primers specific for EpCAM2. Also provided is a composition comprising a pair of primers specific for Map7 and a pair of primers specific for EpCAM2. Also provided is a composition comprising a pair of primers specific for E-cadherin and a pair of primers specific for P-cadherin. Primers capable of specifically amplifying the markers of the invention can be used in the methods and compositions provided. Methods of designing and testing additional primers are available to the skilled person using the published sequences of the named markers. Polynucleotides for use as amplification primers or as probes for the present markers are known or can be routinely designed based on the sequences of the genes encoding these markers, which are known in the art.

For example, nucleotide sequence of human cytokeratin 19 is found at GenBank accession no NM_{—}002276. The polypeptide sequences, nucleic acid sequences encoding cytokeratin 19, and the information set forth under GenBank Accession No. NM_{—}002276, are hereby incorporated by reference.

For example, nucleotide sequence of human Map7 is found at GenBank accession no. NM_{—}003980. The polypeptide sequences, nucleic acid sequences encoding Map7, and the information set forth under GenBank Accession No. NM_{—}003980, are hereby incorporated by reference.

For example, nucleotide sequence of human EpCAM2 is found in Chen et al. (Accurate discrimination of pancreatic ductal adenocarcinoma and chronic pancreatitis using multimarker expression data and samples obtained by minimally invasive fine needle aspiration, Int J Cancer. 2007 Apr. 1; 120(7):1511-7), which is hereby incorporated by reference for its reference to the sequence of EpCAM2 (tacstd).

For example, nucleotide sequence of human P-cadherin is found at GenBank accession no. NM_{—}001793. The polypeptide sequences, nucleic acid sequences encoding P-cadherin, and the information set forth under GenBank Accession No. NM_{—}001793 is hereby incorporated by reference.

For example, nucleotide sequence of human E-cadherin is found at GenBank accession no. NM_{—}004360. The polypeptide sequences, nucleic acid sequences encoding P-cadherin, and the information set forth under GenBank Accession No. NM_{—}004360 is hereby incorporated by reference.

A kit is provided, comprising a polynucleotide for use as amplification primer or as a probe for the present markers is provided. The kit can contain any one or more of the listed polynucleotides along with any other primers needed to perform an amplification of one of the target nucleic acids disclosed.

The disclosed methods can use RT-PCR of paraffin-embedded formalin fixed (FFPE). Real-time RT-PCR data were quantified in terms of cycle threshold (Ct) values. Ct values are inversely related to the amount of starting template; high Ct values correlate with low levels of gene expression, whereas low Ct values correlate with high levels of gene expression.

In implementing the present method, reference may optionally be made to a general review of PCR techniques and to the explanatory note entitled “Quantitation of DNA/RNA Using Real-Time PCR Detection” published by Perkin Elmer Applied Biosystems (1999) and to PCR Protocols (Academic Press New York, 1989).

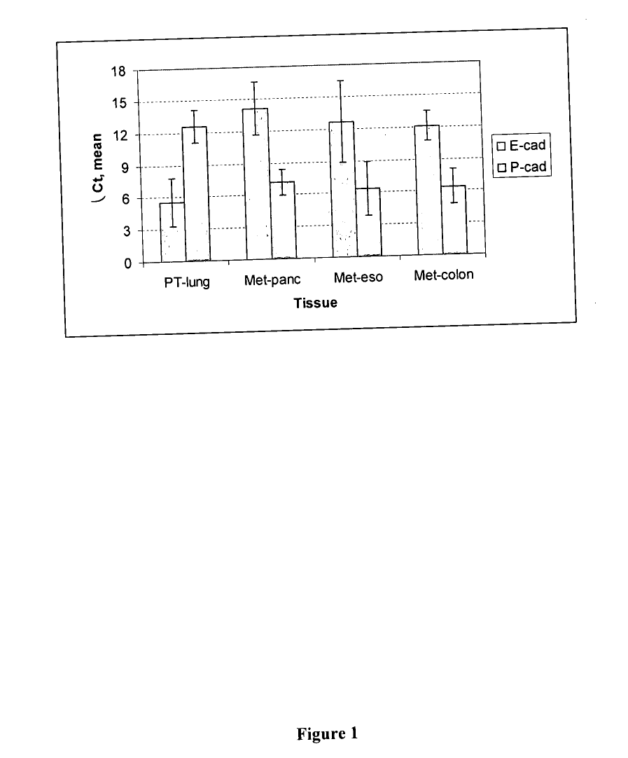

Real-time PCR monitors the fluorescence emitted during the reaction as an indicator of amplicon production during each PCR cycle (ie, in real time) as opposed to the endpoint detection (For example see FIG. 1; Higuchi, 1992; Higuchi, 1993). The real-time progress of the reaction can be viewed in some systems.

The real-time PCR system is based on the detection of a fluorescent reporter (Lee, 1993; Livak, 1995). This signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. The higher the starting copy number of the nucleic acid target, the sooner a significant increase in fluorescence is observed.

A fixed fluorescence threshold is set significantly above the baseline that can be altered by the operator. The parameter CT (threshold cycle) is defined as the cycle number at which the fluorescence emission exceeds the fixed threshold.

There are three main fluorescence-monitoring systems for DNA amplification (Wittwer, 1997(a)): (1) hydrolysis probes; (2) hybridising probes (see Hybridisation Probe Chemistry, incorporated herein by reference for its teaching of fluorescence monitoring systems); and (3) DNA-binding agents (Wittwer, 1997; van der Velden, 2003, incorporated herein for their teaching of DNA-binding agents). Hydrolysis probes include TaqMan™ probes (Heid et al, 1996, incorporated herein by reference for its teaching of hydrolysis probes), molecular beacons (Mhlanga, 2001; Vet, 2002; Abravaya, 2003; Tan, 2004; Vet & Marras, 2005, incorporated herein by reference for their teaching of molecular beacons) and scorpions (Saha, 2001; Solinas, 2001; Terry, 2002, incorporated herein by reference for their teaching of scorpions). They use the fluorogenic 5′ exonuclease activity of Taq polymerase to measure the amount of target sequences in cDNA samples (see also Svanvik, 2000, incorporated herein by reference for its teaching of light-up probes).

TaqMan™ probes are oligonucleotides longer than the primers (20-30 bases long with a Tm value of 10oC higher) that contain a fluorescent dye usually on the 5′ base, and a quenching dye typically on the 3′ base. When irradiated, the excited fluorescent dye transfers energy to the nearby quenching dye molecule (this is called FRET=Förster or fluorescence resonance energy transfer) (Hiyoshi, 1994; Chen, 1997). Thus, the close proximity of the reporter and quencher prevents detection of any fluorescence while the probe is intact. TaqMan™ probes are designed to anneal to an internal region of a PCR product. When the polymerase replicates a template on which a TaqMan™ probe is bound, its 5′ exonuclease activity cleaves the probe (Holland, 1991). This ends the activity of quencher (no FRET) and the reporter dye starts to emit fluorescence which increases in each cycle proportional to the rate of probe cleavage. Accumulation of PCR products is detected by monitoring the increase in fluorescence of the reporter dye (note that primers are not labelled). TaqMan™ assay uses universal thermal cycling parameters and PCR reaction conditions. Because the cleavage occurs only if the probe hybridises to the target, the origin of the detected fluorescence is specific amplification. The process of hybridisation and cleavage does not interfere with the exponential accumulation of the product. One specific requirement for fluorogenic probes is that there be no G at the 5′ end. A ‘G’ adjacent to the reporter dye can quench reporter fluorescence even after cleavage.

Molecular beacons also contain fluorescent (FAM, TAMRA, TET, ROX) and quenching dyes (typically DABCYL) at either end but they are designed to adopt a hairpin structure while free in solution to bring the fluorescent dye and the quencher in close proximity for FRET to occur. They have two arms with complementary sequences that form a very stable hybrid or stem. The close proximity of the reporter and the quencher in this hairpin configuration suppresses reporter fluorescence. When the beacon hybridises to the target during the annealing step, the reporter dye is separated from the quencher and the reporter fluoresces (FRET does not occur). Molecular beacons remain intact during PCR and must rebind to target every cycle for fluorescence emission. This will correlate to the amount of PCR product available. All real-time PCR chemistries allow detection of multiple DNA species (multiplexing) by designing each probe/beacon with a spectrally unique fluor/quench pair as long as the platform is suitable for melting curve analysis. By multiplexing, the target(s) and endogenous control can be amplified in single tube. For examples, see Bernard, 1998; Vet, 1999; Lee, 1999; Donohoe, 2000; Read, 2001; Grace, 2003; Vrettou, 2004; Rickert, 2004.

With Scorpion probes, sequence-specific priming and PCR product detection is achieved using a single oligonucleotide. The Scorpion probe maintains a stem-loop configuration in the unhybridised state. The fluorophore is attached to the 5′ end and is quenched by a moiety coupled to the 3′ end. The 3′ portion of the stem also contains sequence that is complementary to the extension product of the primer. This sequence is linked to the 5′ end of a specific primer via a non-amplifiable monomer. After extension of the Scorpion primer, the specific probe sequence is able to bind to its complement within the extended amplicon thus opening up the hairpin loop. This prevents the fluorescence from being quenched and a signal is observed.

Another alternative is the double-stranded DNA binding dye chemistry, which quantitates the amplicon production (including non-specific amplification and primer-dimer complex) by the use of a non-sequence specific fluorescent intercalating agent (SYBR-green I or ethidium bromide). It does not bind to ssDNA. SYBR green is a fluorogenic minor groove binding dye that exhibits little fluorescence when in solution but emits a strong fluorescent signal upon binding to double-stranded DNA (Morrison, 1998). Disadvantages of SYBR green-based real-time PCR include the requirement for extensive optimisation. Furthermore, non-specific amplifications require follow-up assays (melting point curve or dissociation analysis) for amplicon identification (Ririe, 1997). The method has been used in HFE-C282Y genotyping (Donohoe, 2000). Another controllable problem is that longer amplicons create a stronger signal (if combined with other factors, this may cause CCD camera saturation, see below). Normally SYBR green is used in singleplex reactions, however when coupled with melting point analysis, it can be used for multiplex reactions (Siraj, 2002).

The threshold cycle or the CT value is the cycle at which a significant increase in ΔRn is first detected. ΔRn is the difference in the fluorescence detected between the measured fluorescence of the background noise and the detected fluorescence of the sample to be analyzed. The threshold cycle is when the system begins to detect the increase in the signal associated with an exponential growth of PCR product during the log-linear phase. This phase provides the most useful information about the reaction (certainly more important than the end-point). The slope of the log-linear phase is a reflection of the amplification efficiency. The efficiency (Eff) of the reaction can be calculated by the formula: Eff=10(−1/slope)−1. The efficiency of the PCR should be 90-110% (−3.6>slope>−3.1). A number of variables can affect the efficiency of the PCR. These factors include length of the amplicon, secondary structure and primer quality. Although valid data can be obtained that fall outside of the efficiency range, the real time PCR should be further optimised or alternative amplicons designed. For the slope to be an indicator of real amplification (rather than signal drift), there has to be an inflection point. This is the point on the growth curve when the log-linear phase begins. It also represents the greatest rate of change along the growth curve. (Signal drift is characterised by gradual increase or decrease in fluorescence without amplification of the product.) The important parameter for quantitation is the CT. The higher the initial amount of genomic DNA, the sooner accumulated product is detected in the PCR process, and the lower the CT value. The threshold should be placed above any baseline activity and within the exponential increase phase (which looks linear in the log transformation). Some software allows determination of the cycle threshold (CT) by a mathematical analysis of the growth curve. This provides better run-to-run reproducibility. Besides being used for quantitation, the CT value can be used for qualitative analysis as a pass/fail measure.

In some aspects of the real time PCR method disclosed, multiplex TaqMan™ assays can be performed with ABI instruments using multiple dyes with distinct emission wavelengths. Available dyes for this purpose are FAM, TET, VIC and JOE (the most expensive). TAMRA is reserved as the quencher on the probe and ROX as the passive reference. For best results, the combination of FAM (target) and VIC (endogenous control) is recommended (they have the largest difference in emission maximum) whereas JOE and VIC should not be combined. It is important that if the dye layer has not been chosen correctly, the machine will still read the other dye's spectrum. For example, both VIC and FAM emit fluorescence in a similar range to each other and when doing a single dye, the wells should be labelled correctly. In the case of multiplexing, the spectral compensation for the post run analysis should be turned on (on ABI 7700: Instrument/Diagnostics/Advanced Options/Miscellaneous). Activating spectral compensation improves dye spectral resolution.

In addition, the real-time PCR reaction can be carried out in a wide variety of platforms including, but not limited to ABI 7700 (ABI), the LightCycler (Roche Diagnostics), iCycler (RioRad), DNA Engine Opticon ContinuousFluorescence Detection System (MJ Research), Mx400 (Stratagene), Chimaera Quantitative Detection System (Thermo Hybaid), Rotor-Gene 3000 (Corbett Research), Smartcycler (Cepheid), or the MX3000P format (Stratagene).

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR. It is understood that in certain embodiments, the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

The polynucleotides (primers or probes) can comprise the usual nucleotides consisting of a base moiety, a sugar moiety and a phosphate moiety, e.g., base moiety—adenin-9-yl (A), cytosin-1-yl (C), guanin-9-yl (G), uracil-1-yl (U), and thymin-1-yl (T); sugar moiety—ribose or deoxyribose, and phosphate moiety—pentavalent phosphate. They can also comprise a nucleotide analog, which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. The polynucleotides can contain nucleotide substitutes which are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the target gene typically will be used to produce an amplified DNA product that contains a region of the target gene or the complete gene. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The nucleic acids, such as the oligonucleotides to be used as primers, can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein and nucleic acid molecules can be made using known methods such as those described by Nielsen et al., Bioconjug. Chem. 5:3-7 (1994).

The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997), incorporated herein by reference.

Disclosed are chips, for example microarray chips, where at least one address is a sequence or part of a sequence set forth in any of the nucleic acid sequences disclosed herein. For example, the chip can contain a probe for a nucleic acid encoding cytokeratin 19, P-cadherin or E-cadherin or any combination thereof.

Therefore, provided herein is an array comprising a substrate having a plurality of addresses, wherein each address comprises a capture probe that specifically binds under stringent conditions a nucleic acid encoding cytokeratin 19, EpCAM2, Map7, P-cadherin or E-cadherin or to a complement thereof. In a specific aspect the nucleic acids bound by the capture probe can be a cytokeratin 19-specific nucleic acid, an EpCAM2-specific nucleic acid, a Map7-specific nucleic acid, a P-cadherin-specific nucleic acid or an E-cadherin-specific nucleic acid. A nucleic acid bound by the capture probe of each address is unique among the plurality of addresses.

As used herein, “stringent conditions” refers to the washing conditions used in a hybridization protocol. In general, the washing conditions should be a combination of temperature and salt concentration chosen so that the denaturation temperature is approximately 5-20° C. below the calculated Tm of the nucleic acid hybrid under study. The temperature and salt conditions are readily determined empirically in preliminary experiments in which samples of reference DNA immobilized on filters are hybridized to the probe or protein coding nucleic acid of interest and then washed under conditions of different stringencies. The Tm of such an oligonucleotide can be estimated by allowing 2° C. for each A or T nucleotide, and 4° C. for each G or C. For example, an 18 nucleotide probe of 50% G+C would, therefore, have an approximate Tm of 54° C. Stringent conditions are known to one of skill in the art. See, for example, Sambrook et al. (2001). An example of stringent wash conditions is 4×SSC at 65° C. Highly stringent wash conditions include, for example, 0.2×SSC at 65° C.

To create arrays, single-stranded polynucleotide probes can be spotted onto a substrate in a two-dimensional matrix or array. Each single-stranded polynucleotide probe can comprise at least 6, 7, 8, 9, 10, 11, 12, 13, 14, 15, 16, 17, 18, 19, 20, 25, or 30 or more contiguous nucleotides selected from the nucleotide coding sequences of a plurality of markers, for example the markers cytokeratin 19, P-cadherin or E-cadherin. The substrate can be any substrate to which polynucleotide probes can be attached including, but not limited to, glass, nitrocellulose, silicon, and nylon. Polynucleotide probes can be bound to the substrate by either covalent bonds or by non-specific interactions, such as hydrophobic interactions. Techniques for constructing arrays and methods of using these arrays are described in EP No. 0799 897; PCT No. WO 97/29212; PCT No. WO 97/27317; EP No. 0 785 280; PCT No. WO 97/02357; U.S. Pat. Nos. 5,593,839; 5,578,832; EP No. 0728 520; U.S. Pat. No. 5,599,695; EP No. 0721 016; U.S. Pat. No. 5,556,752; PCT No. WO 95/22058; and U.S. Pat. No. 5,631,734. Commercially available polynucleotide arrays, such as Affymetrix GeneChip™, can also be used. Use of the GeneChip™ to detect gene expression is described, for example, in Lockhart et al., Nature Biotechnology 14:1675 (1996); Chee et al., Science 274:610 (1996); Hacia et al., Nature Genetics 14:441, 1996; and Kozal et al., Nature Medicine 2:753, 1996.

Tissue samples can be treated to form single-stranded polynucleotides, for example, by heating or by chemical denaturation, as is known in the art. The single-stranded polynucleotides in the tissue sample can then be labeled and hybridized to the polynucleotide probes on the array. Detectable labels which can be used include, but are not limited to, radiolabels, biotinylated labels, fluorophors, and chemiluminescent labels. Double stranded polynucleotides, comprising the labeled sample polynucleotides bound to polynucleotide probes, can be detected once the unbound portion of the sample is washed away. Detection can be visual or with computer assistance.

Provided is a method of detecting the markers and marker ratios disclosed herein by measuring or detecting the marker protein using an antibody to the protein or other molecule that can capture or detect the marker protein. In most instances the capturing/detecting molecule will be specific for the protein.

Disclosed herein are kits that are drawn to reagents that can be used in practicing the methods disclosed herein. The kits can include any reagent or combination of reagents discussed herein or that would be understood to be required or beneficial in the practice of the disclosed methods. For example, the kits could include primers to perform the amplification reactions described, as well as the buffers and enzymes required to use the primers as intended. For example, disclosed is a kit for assessing a subject's risk for cancer metastasis, comprising any one or more of the oligonucleotides probes for cytokeratin 19, P-cadherin or E-cadherin. The kit can include instructions for using the reagents described in the methods disclosed herein.

Having provided a means for staging cancer based on the overexpression of certain markers, the invention allows for more accurate staging of cancers than current techniques allow. In contrast to the standard method of staging cancer, which relies on histopathologic detection of cancer in the lymph nodes (in combination with primary tumor size and the presence or absence of cancer elsewhere in the body), the detection of markers as taught in the present invention is more sensitive, and thus, more accurate. As shown herein, the overexpression of certain markers or combinations of markers is indicative of a later stage of cancer than was determined using the standard, histopathology-based methods. The present RT-PCR methodology provides valuable prognostic information which allows the clinician to make more informed adjuvant therapy decisions. Thus, the improved information about the stage of a patient's cancer provided by the present methods can be used to tailor a treatment regimen to that patient, increasing the likelihood of improved outcome.

The present method can be used to test paraffin embedded tissues by PCR. These tissues may be from patients currently showing no sign of metastasis according to the usual clinical methods. Thus, testing of the paraffin samples of these patients may be used to inform the doctor and patient of undetected metastasis or the likelihood of later relapse. This method also permits the use of PCR to detect metastasis in specimens that are prepared for the standard histopathologic analysis.

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in C or is at ambient temperature, and pressure is at or near atmospheric.

### EXAMPLES

In the present study, we two examples in which expression levels of P-cadherin and another gene are prognostic indicators of clinical outcome.

### Example 1

### P-Cadherin/E-Cadherin Ratio

Expression levels of P-cadherin and E-cadherin were measured in several adenocarcinoma tumors (n=20) and in metastatic tissues derived from pancreatic, colon, and esophageal cancer patients. The ratio of P to E was low in lung primary tumors, and high in all metastatic tissues. See FIG. 1.

### Example 2

### P-Cadherin/CK19 Ratio as a Predictor of Recurrence

Primary tumors were obtained from 20 early stage (I or II) who died within two years of surgery (n=9; these patients are identified in the figure with a “1” in the column labeled “Endpoint”) or survived 4 or more years following surgery (n=11; these patients are identified in the figure with a “0” in the column labeled “Endpoint”). Expression levels of 14 genes (listed as 1-14) were measured in the primary tumors. The ratio (listed as “ratio”) of P-cadherin (listed as gene 8) to CK19 (listed as gene 10) correlated highly with clinical outcome. See table below.

| | | End | | | | | | | | | | | | | | | |
|---|---|---|---|---|---|---|---|---|---|---|---|---|---|---|---|---|---|
| Pt# | Stage | point | Ratio | 1 | 2 | 3 | 4 | 5 | 6 | 7 | 8 | 9 | 10 | 11 | 12 | 13 | 14 |
| 27 | IIB | 1 | 2.2 | 4.1 | 19.3 | 5.2 | 7.4 | 4.4 | 7.1 | 9.6 | 14.4 | 7.3 | 16.6 | 2.2 | 8.0 | 5.6 | 6.8 |
| 2 | IIB | 1 | 0.7 | 11.0 | 14.5 | 5.1 | 6.9 | 1.2 | 5.9 | 7.3 | 12.8 | 11.0 | 13.5 | 4.9 | 7.0 | 5.3 | 5.3 |
| 14 | IB | 1 | 0.0 | 5.6 | 15.3 | 4.9 | 7.8 | 4.0 | 8.3 | 8.2 | 13.6 | 8.6 | 13.7 | 4.4 | 8.5 | 7.4 | 6.5 |
| 8 | IIB | 1 | −0.4 | 4.7 | 10.4 | 3.6 | 8.3 | 4.1 | 4.4 | 10.0 | 11.8 | 15.8 | 11.4 | 2.7 | 8.1 | 6.4 | 4.3 |
| 5 | IA | 1 | −1.1 | 4.7 | 13.5 | 4.1 | 6.9 | 4.5 | 7.0 | 8.1 | 13.5 | 5.6 | 12.4 | 2.1 | 7.2 | 6.5 | 5.8 |
| 15 | IA | 1 | −1.2 | 9.2 | 14.7 | 6.8 | 7.6 | 6.8 | 7.4 | 12.5 | 14.7 | 13.2 | 13.5 | 6:5 | 8.2 | 7.6 | 6.3 |
| 19 | IA | 0 | −1.8 | 5.7 | 15.4 | 3.9 | 6.3 | 4.5 | 5.7 | 7.1 | 13.2 | 12.4 | 11.4 | 4.2 | 7.3 | 4.7 | 5.5 |
| 16 | IIB | 0 | −2.1 | 9.3 | 10.5 | 3.9 | 8.2 | 6.4 | 6.0 | 9.5 | 14.7 | 15.8 | 12.6 | 4.0 | 8.0 | 7.7 | 4.5 |
| 26 | IA | 0 | −2.4 | 6.3 | 17.5 | 3.7 | 8.0 | 4.1 | 7.2 | 7.9 | 13.5 | 11.1 | 11.1 | 3.0 | 7.1 | 5.5 | 5.2 |
| 22 | IIB | 1 | −2.4 | 4.4 | 8.4 | 3.4 | 6.9 | 2.2 | 5.8 | 7.8 | 12.8 | 7.5 | 10.4 | 1.2 | 6.8 | 5.3 | 4.7 |
| 21 | IB | 0 | −2.7 | 4.4 | 17.7 | 4.9 | 7.2 | 4.7 | 6.3 | 7.5 | 13.9 | 14.1 | 11.3 | 6.9 | 8.1 | 6.8 | 5.7 |
| 29 | IB | 0 | −2.8 | 3.3 | 18.3 | 3.2 | 7.4 | 3.0 | 4.8 | 7.4 | 11.3 | 10.4 | 8.5 | 2.4 | 6.8 | 5.9 | 4.8 |
| 12 | IB | 1 | −2.8 | 4.7 | 14.7 | 2.9 | 6.8 | 1.3 | 5.0 | 6.5 | 12.6 | 10.6 | 9.8 | 3.2 | 5.5 | 5.1 | 3.4 |
| 17 | IIB | 1 | −3.6 | 14.6 | 14.7 | 6.3 | 8.3 | 7.8 | 7.2 | 10.2 | 13.7 | 16.0 | 10.1 | 5.1 | 7.8 | 7.3 | 5.8 |
| 11 | IA | 0 | −3.6 | 6.7 | 15.5 | 2.7 | 6.7 | 1.9 | 2.8 | 9.7 | 11.0 | 8.8 | 7.4 | 1.8 | 5.9 | 5.0 | 2.3 |
| 24 | IA | 0 | −4.1 | 5.3 | 9.5 | 4.9 | 8.7 | 6.0 | 7.5 | 7.6 | 13.8 | 8.1 | 9.7 | 3.8 | 9.4 | 7.4 | 7.1 |
| 13 | IA | 0 | −4.7 | 8.1 | 11.2 | 4.8 | 7.4 | 5.0 | 7.8 | 10.1 | 13.2 | 9.0 | 8.5 | 4.5 | 7.1 | 6.8 | 7.6 |
| 4 | IB | 0 | −5.1 | 8.2 | 17.0 | 8.5 | 6.8 | 8.0 | 8.8 | 11.4 | 14.0 | 14.9 | 9.0 | 8.0 | 8.7 | 8.3 | 7.3 |
| 25 | IA | 0 | −5.1 | 4.9 | 8.8 | 4.7 | 9.3 | 5.0 | 6.9 | 7.6 | 14.1 | 5.4 | 9.0 | 2.4 | 8.3 | 5.6 | 6.4 |
| 20 | IIB | 0 | −7.6 | 8.6 | 10.4 | 7.2 | 8.1 | 0.1 | 7.7 | 8.3 | 10.3 | 10.3 | 2.7 | 7.8 | 5.0 | 6.3 | 3.9 |

### Example 3

### CK19/EpCAM2 Ratio is a Simple and Accurate Prognostic Indicator of Clinical Outcome in Early Stage Adenocarcinoma

Methods Summary Twenty-two prognostic genes for the metastatic phenotype were identified through cDNA microarray analysis of cancer cell lines and bioinformatics analysis. Expression levels of a subset of these genes (n=13) were measured by real-time RT-PCR in FFPE primary adenocarcinoma from patients who recurred within 2 years (n=9) and who did not recur (n=11). ROC curve analysis was performed to establish prognostic values of single genes, and then the most informative gene was combined with the remaining genes to determine if there was a particular pair that yielded high diagnostic accuracy.

Results Summary: ROC curve analysis of the single genes revealed that high expression of CK19 was associated with non-recurrence (AUC=0.859, CI=0.651-0.970). The CK19/EpCAM2 gene ratio had the most reproducible prognostic accuracy. A Kaplan Meier survival analysis was generated from the CK19/EpCAM2 ratio and resulted in highly significant curves as a function of marker positivity (p=0.0007; HR=10.7).

Conclusions: This Example provides evidence that the CK19/EpCAM2 ratio is a simple and accurate prognostic indicator of clinical outcome in early stage adenocarcinoma of the lung. The gene pair with the second highest prognostic accuracy for disease recurrence in early stage NSCLC was CK19/P-cadherin. Adjuvant therapy can be targeted to this high risk group to improve survival, and vice versa, not targeted to those at low risk to avoid the toxicity.

### Materials and Methods

Identification of 15 highly expressed genes in NSCLC cell lines. Expression levels of 22,283 gene transcripts were determined on oligonucleotide microarrays using RNA prepared from four NSCLC cell lines [CRL 5807 (bronchoalveolar carcinoma), CRL 5876 (adenocarcinoma derived from metastatic lymph node), A549 (adenocarcinoma), and HTB 177 (large cell carcinoma)], as well as from a pool of 4 normal cervical lymph nodes. Eight μg of total RNA per sample was used. First and 2nd strand cDNA synthesis, double stranded cDNA cleanup, biotin-labeled cRNA synthesis, cleanup and fragmentation were performed according to protocols in the Affymetrix GeneChip Expression Analysis technical manual (Affymetrix). Microarray analysis was performed by the DNA Microarray and Bioinformatics Core Facility at the Medical University of South Carolina using U133 A GeneChips (Affymetrix). Fluorescent images of hybridized microarrays were obtained by using a HP GeneArray scanner (Affymetrix). For normalization, the microarray office suite was used such that all fluorescence values were multiplied by a factor that resulted in a mean fluorescent score for all genes equal to 150. Data for normal lymph nodes were obtained from a previous study.⁸ All microarray results were imported into single Microsoft Excel file. The first algorithm in the selection of highly expressed genes involved elimination of genes from NSCLC cell lines that were expressed in normal lymph nodes [n=11,326; 50.8% of total (22,283)]. Of the remaining 10,957 genes, those that were detected in at least 2 NSCLC cell lines were first selected (n=1731; 7.7% of total). Following this round, genes whose mean fluorescence in all cell lines were >500 fluorescence units were selected (n=91; 0.41% of total). The final group of 91 genes was sorted according to mean cell line fluorescence/mean fluorescence of normal lymph nodes, and the 15 top genes were selected. (Table 1).

Bioinformatics analysis to identify potentially prognostic genes in NSCLC. Of the 15 most highly expressed genes identified by cDNA microanalysis, it was hypothesized that some of them were also expressed in other cancers, while some genes were specific for NSCLC. To identify genes that were highly expressed in other cancers, the on-line Comparative Genome Anatomy Project (CGAP) NCI 60 gene expression database (URL=http://cgap.nci.nih.gov) was queried using all 15 genes. The output of a given query consists of a list of 10 genes whose expression levels are most highly correlated with the query sequence. Using the output of each gene, a correlation map was constructed such that the appearance of a gene on the map required 1) direct contact with one of the 15 highly expressed genes, 2) contacts with at least two genes, 3) that the correlation coefficient of any two genes must have a p value <8×10⁻⁶, 4) that the relevant gene must be overexpressed in the CGAP SAGE dataset in at least two cancers (with respect to normal tissue), and 5) that expression of the relevant gene must be at least 16-31 tags/200,000 sequenced tags in at least one cancer tissue. Genes identified from the first set of queries were used as query in a reiterative round of interrogation (data mining).

The correlation map obtained using this bioinformatics data mining approach contained a total of 22 genes (FIG. 2). Seven of the 22 genes (AGR2, Map 7, S100P, CK19, EpCAM1, EpCAM2, P-cadherin) were derived from the list of 15 most highly expressed genes and are referred to as the Primary prognostic genes (underlined in FIG. 2). The remaining 15 genes identified from this bioinformatics approach are referred to as the Secondary prognostic genes (italicized in FIG. 2).

Identification of genes of prognostic value in early stage NSCLC adenocarcinoma patients. To determine whether the genes described above had potential prognostic value, the expression levels were measured by real-time RT-PCR in paraffin-embedded formalin fixed (FFPE) primary tumors of adenocarcinoma patients who recurred within two years (poor outcome group A; n=9) and who survived disease-free longer than four years (good outcome group B; n=11). Group A patients included 2 with stage IA, 2 stage IB, and 5 stage IIB. Group B patients included 5 with stage IA, 3 stage IB, and 3 stage IIB. Genes analyzed included the seven primary prognostic genes, six secondary prognostic genes (Sprint 2, Esx, CEA6, Ma12, GPX2, E-cadherin) as well as μPAR, a gene whose expression has previously been shown to be associated with multiple cancers. The initially results were obtained blinded as to the clinical outcome.

Real-time reverse transcription-PCR of formalin-fixed paraffin-embedded samples was performed following the method of Sprecht et al.⁹ A 50-μm section was cut from tissue blocks for mRNA extraction. For isolation of RNA, paraffin-embedded tissue sections were deparaffinized twice with 1 mL of xylene at 37° C. or room temperature for 10 minutes. The pellet was subsequently washed with 1 mL of 100%, 90%, and 70% of ethanol and air-dried at room temperature for 2 hours. The pellet was resuspended in 200 μL of RNA lysis buffer [2% lauryl sulfate, 10 mmol/L Tris-HCI (pH 8.0), and 0.1 mmol/L EDTA] and 100 μg of proteinase K and incubated at 60° C. for 16 hours. RNA was extracted using 1 mL of phenol/chloroform (5:1) solution (Sigma, St. Louis, Mo.). The aqueous layer containing RNA was transferred to a new 1.5 mL tube. Phenol/chloroform extraction was done a total of three times. RNA was precipitated with an equal volume of isopropanol, 0.1 volume of 3 mol/L sodium acetate, and 100 μg of glycogen at −20° C. for 16 hours. After centrifugation at 12,000 rpm for 15 minutes (4° C.), the RNA pellet was washed with 70% of ethanol and air-dried at room temperature for 2 hours. Finally, the pellet was dissolved in 12 μL of DEPC water. cDNA synthesis was performed using a panel of truncated gene-specific primers. Real-time RT-PCR was performed on a PE Biosystems Gene Amp® 7300 or 7500 Sequence Detection System (Foster City, Calif.). With the exception of the SYBR Green I master mix (purchased from Qiagen, Valencia, Calif.), all reaction components were purchased from PE Biosystems. Standard reaction volume was 10 μl and contained 1×SYBR RT-PCR buffer, 3 mM MgCl2, 0.2 mM each of dATP, dCTP, dGTP, 0.4 mM dUTP, 0.1 U UngErase enzyme, 0.25 U AmpliTaq Gold, 0.35 μl cDNA template, and 50 nM of oligonucleotide primer. Initial steps of RT-PCR were 2 min at 50° C. for UNGerase activation, followed by a 10-min hold at 95° C. Cycles (n=40) consisted of a 15 sec melt at 95° C., followed by a 1 min annealing/extension at 60° C. The final step was a 60° C. intubation for 1 min. All reactions were performed in triplicate. Threshold for cycle of threshold (Ct) analysis of all samples was set at 0.5 relative fluorescence units.

Gene expression values were quantified as ΔCt values, which were obtained by subtracting the Ct value of an internal reference control gene (β2-microglobulin, B2M) from the gene of interest. Ct values are inversely proportional to gene expression levels and are based on log2 scale.

The results were internally validated by repeating the real-time RT-PCR process using a new section cut from tissue blocks of the primary tumor. Variability of tumor quantity on the sections was minimized by H&E comparison performed by a pathologist. A cross-validation procedure was used to determine if the results were sensitive to the samples included. A leave-one-out procedure was used where each sample was systemically removed and the data reanalyzed.

Statistical Analysis. To assess for prognostic accuracy, ROC curve analysis was performed on the individual genes normalized to B2M (Med Calc software). Prognostic gene combinations were tested by subtracting ΔCt values generated by RT-PCR analysis. Subtraction of ΔCt values (ΔΔCt) is equivalent to a mathematical division. In the text, the ΔCtgene A-ΔCtgene B calculation is abbreviated as a gene expression ratio. The value of the two-gene prognostic assay was further assessed by Kaplan Meier survival analysis.

### Results

A primary tumor's ability to metastasize requires many genetic events. The correlation map illustrated in FIG. 2 resulted from a unique bioinformatics analysis that led to a set of genes that had specific structured connections based on a query of 15 genes over-expressed in four lung cancer cell lines. Of the 22 identified genes, seven were in the original query set and were labeled primary prognostic genes. These genes combined with 6 of the most frequently expressed remaining 16 secondary genes constituted the study's test gene set in patients with adenocarcinoma of the lung.

AUC (area under the curve) values for the primary and secondary genes are shown in Table 2. ROC curve analysis of the individual genes revealed that high expression of CK19 was associated with non-recurrence (≧4 years) (AUC=0.859; 95% CI=0.651-0.970); whereas high expression of EpCAM2 was associated with disease recurrence within two years (AUC=0.606; 95% CI—0.366-0.813).

To determine whether the prognostic accuracy of CK19 could be improved by combining it with another gene whose overexpression might be necessary for the metastatic phenotype and therefore low expression be favorable, the mean ΔCt values of individual genes as determined by real-time RT-PCR analysis were subtracted from ΔCtCK19. For all potential CK19/gene X combinations, the ratio of CK19/EpCAM2 yielded the highest prognostic accuracy as determined by AUC measurements. (Table 3) This observation provided evidence that EpCAM2 is a “bad” gene. The CK19/EpCAM2 expression ratio, which was derived from the mean of two experiments, also performed well when data were analyzed from individual experiments. In the first and second experiments, the prognostic accuracy of the CK19/EpCAM2 expression ratio as determined by AUC analysis was 0.91 (95% CI=0.69-0.99) and 0.84 (95% CI=0.56-0.97), respectively. Of further note is the observation that of the 12 stage I adenocarcinoma patients, the prognostic accuracy of the CK19/EpCAM2 expression ratio was 92% (11/12).

The cross-validation procedure found no qualitative differences in inferences. For CK19 alone, the range of AUCs found in the cross-validation analyses was (0.87, 0.92) whereas the AUC when all samples were included was 0.86. Analogous results were found when CK19 was combined with EpCAM2.

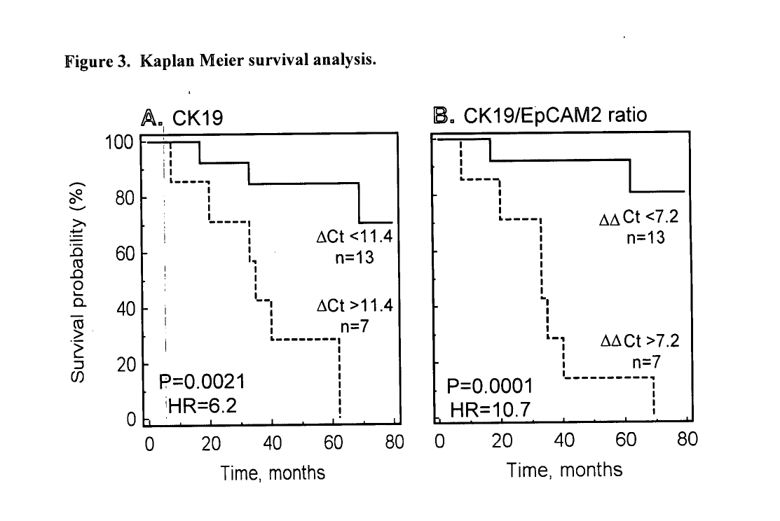

To further assess the value of CK19 unpaired and paired with EpCAM2, a Kaplan Meier survival analysis was performed using data generated from single marker and CK19/gene X analyses. For the single CK19 marker, a ΔCt cutoff of 11.4 was used, which separated the 20 patients into high (ΔCt<11.4; n=13) and low (ΔCt>11.4; n=7) expressing tumors. A log-ranked test indicated that the two curves generated as a function of marker positivity were different at a p value of 0.0021 with a hazard ratio of 6.2. (FIG. 3A) For the CK19/EpCAM2 ratio, a ΔΔCt cutoff of 7.2 was used, which separated the 20 patients into high (ΔΔCt≦7.2; n=13) and low (ΔΔCt>7.2; n=7) groups that correlated with survival. A log-ranked test indicated that the two curves generated as a function of marker positivity were different at a p value of 0.0001 with an associated hazard ratio of 10.7. (FIG. 3B) Kaplan Meier survival analysis of other CK19/gene X pairs are shown in Table 3.

The gene pair with the second highest prognostic accuracy for disease recurrence in early stage NSCLC was CK19/P-cadherin. These results provide evidence that P-cadherin is also a candidate “bad gene” in NSCLC.

CK19 expression levels appear to serve as a reliable indicator of the epithelial content of the primary tumor.

There are several advantages to the technique used in this study. It is a simple two-gene model and uses a technology that is relatively inexpensive and is quickly performed once RNA is extracted. Paraffin-embedded tumor tissue can be screened and an appropriate slide(s) can be sent to a reference laboratory. The technique is amenable to small tissue samples, which may be important if preoperative biopsy directs neoadjuvant therapy.

TABLE 1
Top 15 most highly overexpressed genes in lung cancer cell lines.

| | | | Affymetrix results^{a} | | | | |
|---|---|---|---|---|---|---|---|
| | | | | 2 | 3 | 4 | |
| Gene description | | | 1 | HTB | CRL | CRL | |
| Rank | Gene | Acc. # | A549 | 177 | 5807 | 5876 | Ratio^{b} |
| 1 | AGR2 | NM_006408 | 2124 | 2053 | 3082 | 38 | 960 |
| 2 | S100P | NM_005980 | 242 | 2522 | 2673 | 4819 | 754 |
| 3 | CK19 | NM_002276 | 27 | 935 | 1995 | 810 | 589 |
| 4 | NQO1 | NM_000903 | 1375 | 1858 | 982 | 315 | 404 |
| 5 | MET | NM_000245 | 1420 | 790 | 2429 | 378 | 348 |
| 6 | MAGE-A6 | NM_005363 | 73 | 37 | 3004 | 4475 | 311 |
| 7 | XAGE-1 | NM_020411 | 471 | 2 | 2322 | 3 | 250 |
| 8 | KRTHB1 | NM_002281 | 2822 | 31 | 221 | 3 | 208 |
| 9 | MAGE-A3 | NM_005362 | 116 | 29 | 4055 | 5107 | 178 |
| 10 | MAP7 | NM_003980 | 455 | 466 | 381 | 930 | 116 |
| 11 | AKR1B10 | NM_020299 | 11662 | 10603 | 17 | 75 | 101 |
| 12 | CK7 related | NM_005556 | 537 | 21 | 1319 | 463 | 96 |
| 13 | EpCAM2 | NM_002353 | 2 | 3 | 8146 | 2342 | 94 |
| 14 | EpCAM1 | NM_002354 | 278 | 15 | 4430 | 3244 | 91 |
| 15 | P-cadherin | NM_001793 | 2 | 3 | 1319 | 1274 | 87 |

^{a}Normalized fluorescent values obtained from Affymetrix U133A array data for the indicated cell line.
^{b}Ratio of mean NSCLC cell line data to mean of normal lymph node.

TABLE 2
Recurrence analysis of pilot study using single markers
paired with the internal B2M reference control gene.

Recurrence analysis

| Gene | AUC | 95% CI |
|---|---|---|
| CK19 | 0.859 | 0.631 to 0.970 |
| EpCAM2 | 0.606 | 0.366 to 0.813 |
| AGR2 | 0.596 | 0.357 to 0.805 |
| Esx | 0.566 | 0.329 to 0.782 |
| GPX2 | 0.556 | 0.320 to 0.773 |
| CEA6 | 0.545 | 0.312 to 0.765 |
| E-cadherin | 0.545 | 0.312 to 0.765 |
| EpCAM1 | 0.535 | 0.303 to 0.757 |
| SPINT2 | 0.530 | 0.298 to 0.753 |
| S100P | 0.525 | 0.294 to 0.749 |
| MAL2 | 0.515 | 0.285 to 0.740 |
| P-cadherin | 0.510 | 0.281 to 0.736 |
| Map7 | 0.500 | 0.272 to 0.728 |
| UPAR | 0.470 | 0.247 to 0.702 |

TABLE 3
Recurrence and survival analysis of pilot
study based on CK19/geneX ratios

| | |
|---|---|
| | Kaplan Meier |
| Recurrence analysis | survival analysis |

| geneX | AUC | 95% CI | P-value | HR |
|---|---|---|---|---|
| EpCAM2 | 0.879 | 0.656 to 0.978 | 0.0001 | 10.7 |
| P-cadherin | 0.874 | 0.650 to 0.976 | 0.0003 | 8.13 |
| MAL2 | 0.869 | 0.643 to 0.974 | 0.0004 | 9.24 |
| Esx | 0.742 | 0.501 to 0.908 | 0.0008 | 6.62 |
| Map7 | 0.889 | 0.668 to 0.981 | 0.0013 | 6.24 |
| UPAR | 0.843 | 0.613 to 0.963 | 0.0013 | 6.24 |
| E-cadherin | 0.818 | 0.584 to 0.951 | 0.0013 | 6.25 |
| AGR2 | 0.859 | 0.631 to 0.970 | 0.0098 | 4.69 |
| GPX2 | 0.722 | 0.480 to 0.895 | 0.0184 | 5.12 |
| SPINT2 | 0.848 | 0.619 to 0.965 | 0.0207 | 7.78 |
| EpCAM1 | 0.798 | 0.561 to 0.940 | 0.0207 | 7.78 |
| S100P | 0.732 | 0.490 to 0.901 | 0.0275 | 4.08 |
| CEA6 | 0.732 | 0.490 to 0.901 | 0.0729 | 3.10 |

### Example 4

### Map7/EpCAM2 Ratio is a Simple and Accurate Prognostic Indicator of Clinical Outcome in Early Stage Adenocarcinoma

A study was performed with the samples from location A (MUSC data set table below) which were treated with DNAse. Data were analyzed using Kaplan Meier curves and a summary is shown below. The P values shown represent the probability that the two curves generated using the marker pair are the same (i.e., the lower the P value, the better the marker). The column headed “Hazard” represents the Hazard Ratio; it is the odds ratio that a patient with a high marker result will have a worse clinical outcome compared to a patient with a low marker result. Although CK19/EpCAM2 still proved to be valuable in the second analysis, the expression ratio of Map7/EpCAM2 was the most informative.

To further investigate the prognostic value of the makers, a further experiment was performed using DNAse treated samples obtained from location B (JAX data set table below). The results indicated that the Map7/EpCAM2 gene ratio had high prognostic value, indicating that this gene pair is the most reliable and accurate in the data sets.

| | | | |
|---|---|---|---|
| Good | Bad | P | Hazard |

JAX data set (DNAse)

| | | | |
|---|---|---|---|
| MAP7 | XAG | <0.0001 | 6.50 |
| MAP7 | EpCAM2 | <0.0001 | 4.95 |
| MAP7 | CDH3 | 0.0001 | 4.58 |
| MAP7 | Mal2 | 0.0010 | 3.55 |
| EPCAM2 | CDH3 | 0.0013 | 3.45 |
| CEA | S100P | 0.0055 | 3.43 |
| MAP7 | CK19 | 0.0021 | 3.20 |
| MAP7 | GPX2 | 0.0047 | 3.17 |
| Elf3 | GPX2 | 0.0199 | 2.82 |
| MAP7 | S100P | 0.0147 | 2.60 |
| CDH1 | CDH3 | 0.0169 | 2.55 |
| XAG | S100P | 0.0173 | 2.50 |
| MAP7 | CDH1 | 0.0239 | 2.47 |

MUSC Data set (DNAse)

| | | | |
|---|---|---|---|
| MAP7 | EpCAM2 | 0.0135 | 8.64 |
| CK19 | CEA | 0.0257 | 5.04 |
| Elf3 | EpCAM2 | 0.0264 | 4.17 |
| CK19 | EpCAM2 | 0.0496 | 3.59 |

### REFERENCES

1. Mountain CF. Revisions in the international system for staging lung cancer. Chest 1997; 111:1710-17.
2. The International Adjuvant Lung Cancer Trial Collaborative Group. Cisplatin-based adjuvant chemotherapy in patients with completely resected non-small-cell lung cancer. N Engl J Med 2004; 350:351-60.
3. Winton T, Livingston R, Johnson D, Rigas J, Johnston M, Butts C, et al. Vinorelbine plus cisplatin vs. observation in resected non-small cell lung cancer. N Engl J Med 2005; 352:2589-97.
4. Strauss G, Herndon J, Maddaus M, Johnstone D W, Johnson E A, Watson D M, et al. Adjuvant chemotherapy in stage IB non-small cell lung cancer (NSCLC): update of cancer and leukemia group B (CALGB) protocol 9633. Proc Am Soc Clin Oncol 2006; 24:365s.
5. Pignon J, Tribodet H, Scagliotti G, Douillard J Y, Shepherd F A, Stephens R J, et al. Lung adjuvant cisplatin evaluation (LACE): a pooled analysis of five randomized clinical trials including 4,584 patients. Proc Am Soc Clin Oncol 2006; 24:366s.
6. Potti A, Mukherjee S, Petersen R, Dressman H K, Bild A, Koontz J, et al. A genomic strategy to refine prognosis in early-stage non-small cell lung cancer. N Engl J Med 2006; 355:570-80.
7. Bertucci F, Finetti P, Cervera N, Maraninchi D, Viens P, Birnbaum D. Gene expression profiling and clinical outcome in breast cancer. J Integrative Biol 2006; 10:429-43.
8. Mikhitarian K, Gillanders W E, Almeida J S, Herbert-Martin R, Varela J C, Metcalf J S, et al. An innovative microarray strategy identifies informative molecular markers for the detection of micrometastatic breast cancer. Clin Cancer Res 2005; 11:C-704.
9. Sprecht K, Richter T, Mueller U, Walch A, Werner M, Hofler H. Quantitative gene expression analysis in microdissected archival formalin-fixed and paraffin-embedded tumor tissue. Am J Pathol 2001; 158:419-29.
10. Segal E, Friedman N, Koller D, Regev A. A module map showing conditional activity of expression modules in cancer. Nature Genetics 2004; 36:1090-98.
11. Brundage M D, Davies D, Mackillop W J. Prognostic factors in non-small cell lung cancer: a decade of progress. Chest 2002; 122:1037-57.
12. D'Amico T A, Massey M, Herndon J E, Moore M-B, Harpole D H. A biologic risk model for stage I lung cancer: immunohistochemical analysis of 408 patients with the use of ten molecular markers. J Thorac Cardiovasc Surg 1999; 117:736-43.
13. Garber M E, Troyanskaya O G, Schluens K, Petersen S, Taesher Z, Pacyna-Gengelbach M, et al. Diversity of gene expression in adenocarcinoma of lung. Pro Natl Acad Sci USA 2001; 98:13784-89.
14. Chen C, Gharib T G, Huang C-C, Kuick R, Thomas D G, Shedden K A, Misek D E, et al. Protein profiles associated with survival in lung adenocarcinoma. Proc Natl Acad Sci USA 2003; 100:13537-42.
15. Bhattacharjee A, Richards W G, Staunton J, Li C, Monti S, Vasa P, et al. Classification of human lung carcinomas by mRNA expression profiling reveal distinct adenocarcinoma subclasses. Proc Natl Acad Sci USA 2001; 98:13790-95.
16. Wigle D A, Jurisica I, Radulovich N, Pintilie M, Rossant J, Liu N, et al. Molecular profiling of non-small cell lung cancer and correlation with disease-free survival. Cancer Res 2002; 62:3005-8.
17. Yanaihara N, Caplen N, Bowman E, Seike M, Kumamoto K, Yi M, et al. Unique microRNA molecular profiles in lung cancer diagnosis and prognosis. Cancer Cell 2006; 9:189-98.
18. Endoh H, Tomida S, Yatabe Y, Konishi H, Osada H, Tajima K, et al. Prognostic model of pulmonary adenocarcinoma by expression profiling of eight genes as determined by quantitative real-time reverse transcriptase polymerase chain reaction. J Clin Oncol 2004; 22:811-19.
19. Gordon G J, Jensen R V, Hsiao L-L, Gullans S R, Blumenstock J E, Ramaswamy S, et al. Translation of microarray data into clinically relevant cancer diagnostic tests using gene expression ratios in lung cancer and mesothelioma. Cancer Res 2002; 62:4963-67.
20. Chen H Y, Yu S L, Chen C H, Chang G C, Chen C Y, Yuan A, et al. A five gene signature and clinical outcome in non-small cell lung cancer. N Engl J Med 2007; 356:11-20.
21. Kikuchi T, Carbone D P. Proteomic analysis in lung cancer: challenges and opportunities. Respirology 2007; 12:22-8.
22. Ohmachi T. Taneka F, Mimori K, Inoue H, Yanaga K, Mori M. Clinical significance of TROP2 in colorectal cancer. Clin Cancer Res 2006; 12:3057-63.
23. Patel I S, Madan P, Getsios S, Bertrand M M, MacCalman C O. Cadherin switching in ovarian cancer progression. Int J Cancer 2003; 106:172-77.
24. Zou M, Famulski K S, Parhour R S, Baitei E, Al-Mohann F A, Farid N R, et al. Microarray analysis of metastasis-associated gene expression profiling in a murine model of thyroid carcinoma pulmonary metastasis: identification of S100A4 (Mts 1) gene over expression as a poor prognostic marker for thyroid carcinoma. J Clin Endocrinol Metab 2004; 89:61646-54.
25. Taniuchi K, Nakagawa H, Hosokawa M, Nakamura T, Eguchi H, Ohigashi H, et al. Over expressed P-cadherin/CDH3 promotes motility of pancreatic cancer cells by interacting with p120ctn and activating rho-family GTPases. Cancer Res 2005; 65:3092-99.
26. Allard W J, Matera J, Miller M C, Repollet M, Connelly M C, Rao C, et al. Tumor cells circulate in the peripheral blood of all major carcinomas but not in healthy subjects of patients with nonmalignant diseases. Clin Cancer Res 2004; 10:6897-6904.
27. Cristofanilli M, Budd G T, Ellis M J, Stopeck A, Matera J, Miller M C, et al. Circulating tumor cells, disease progression, and survival in metastatic breast cancer. N Engl J Med 2004; 351:781-91.
28. Takeichi, M. Morphogenetic roles of classic cadherins. Curr Opin Cell Biol 7, 619-27 (1995).
29. Cavallaro, U. & Christofori, G. Cell adhesion and signalling by cadherins and Ig-CAMs in cancer. Nat Rev Cancer 4, 118-32 (2004).
30 Chen, W. C. & Obrink, B. Cell-cell contacts mediated by E-cadherin (uvomorulin) restrict invasive behavior of L-cells. J Cell Biol 114, 319-27 (1991).
31 Frixen, U. H. et al. E-cadherin-mediated cell-cell adhesion prevents invasiveness of human carcinoma cells. J Cell Biol 113, 173-85 (1991).
32 Taniuchi, K. et al. Overexpressed P-cadherin/CDH3 promotes motility of pancreatic cancer cells by interacting with p120ctn and activating rho-family GTPases. Cancer Res 65, 3092-9 (2005).
33. Patel, I. S., Madan, P., Getsios, S., Bertrand, M. A. & MacCalman, C. D. Cadherin switching in ovarian cancer progression. Int J Cancer 106, 172-7 (2003).

What is claimed is:

1. A method of identifying a subject at risk for recurrence of adenocarcinoma, comprising determining the ratio of EpCAM2 to CK19 in primary adenocarcinoma tissue from the subject, a high ratio of EpCAM to CK19 M indicating a subject at risk for recurrence.

2. The method of claim 1, wherein a high ratio of EpCAM to CK19 is >128.

3. A method of identifying the presence of metastatic adenocarcinoma tissue in a subject, comprising measuring EpCAM2 and CK19 in primary adenocarcinoma tissue from the subject, a high ratio of EpCAM2 to CK19 indicating the presence of metastatic adenocarcinoma tumor tissue in the subject.

4. A method of identifying a subject at risk for recurrence of adenocarcinoma, comprising determining the ratio of CK19 and P-cadherin in primary adenocarcinoma tissue from the subject, a low ratio of CK19 to P-cadherin indicating a subject at risk for recurrence.

5. The method of claim 4, wherein a low ratio of CK19 to P-cadherin is >0.5.

6. A method of identifying the presence of metastatic adenocarcinoma tissue in a subject, comprising measuring CK19 and P-cadherin in primary adenocarcinoma tissue from the subject, a low ratio of CK19 to P-cadherin indicating the presence of metastatic adenocarcinoma tissue in the subject.

7. A method of identifying a subject at risk for recurrence of adenocarcinoma, comprising determining the ratio of Map7 to EpCAM2 in primary adenocarcinoma tissue from the subject, a ratio of 16:1 indicating a subject at risk for recurrence.

8. A method of identifying a subject at risk for recurrence of adenocarcinoma, comprising determining the ratio of P-cadherin to E-cadherin in primary adenocarcinoma tissue from the subject, a high ratio of P-cadherin to E-cadherin indicating a subject at risk for recurrence.

9. The method of claim 8, wherein a high ratio is from about 5:2 to about 5:3

10. The method of claims 1, wherein the adenocarcinoma is non small cell lung cancer.

11. The method of claims 6, wherein the adenocarcinoma is non small cell lung cancer.

12. The method of claims 7, wherein the adenocarcinoma is non small cell lung cancer.

13. The method of claims 8, wherein the adenocarcinoma is non small cell lung cancer.


SEQUENCE LISTING (8 sequences)

SEQ ID NO: 1 - DNA, 19 bp, Artificial Sequence
  Description of Artificial Sequence note = synthetic construct
aacggcgagctagaggtga

SEQ ID NO: 2 - DNA, 21 bp, Artificial Sequence
  Description of Artificial Sequence note = synthetic construct
ttccgtctcaaacttggttcg

SEQ ID NO: 3 - DNA, 20 bp, Artificial Sequence
  Description of Artificial Sequence note = synthetic construct
aggacaaagaacgccacgaa

SEQ ID NO: 4 - DNA, 21 bp, Artificial Sequence
  Description of Artificial Sequence note = synthetic construct
cacgaccaacggttatgcttc

SEQ ID NO: 5 - DNA, 22 bp, Artificial Sequence
  Description of Artificial Sequence note = synthetic construct
acccgaggagaagaggagtttg

SEQ ID NO: 6 - DNA, 22 bp, Artificial Sequence
  Description of Artificial Sequence note = synthetic construct
gcttctttcccagtgacaagca

SEQ ID NO: 7 - DNA, 21 bp, Artificial Sequence
  Description of Artificial Sequence note = synthetic construct
atcatcgtgaccgaccagaat

SEQ ID NO: 8 - DNA, 21 bp, Artificial Sequence
  Description of Artificial Sequence note = synthetic construct
ggatggagtaagcaaccaccc